(19) United States
(12) Plant Patent Application Publication
(10) Pub. No.: US 2008/0010713 P1
(43) Pub. Date: Jan. 10, 2008

(54) ANTHURIUM PLANT NAMED 'ANTHEPEDI'

(76) Inventor: Jan Van Dijk, Bleiswijk (NL)

Correspondence Address: JONDLE & ASSOCIATES P.C., 858 HAPPY CANYON ROAD SUITE 230, CASTLE ROCK, CO, 80108, US

(21) Appl. No.: 11/482,613

(22) Filed: Jul. 7, 2006

(51) Int. Cl. A01H 5/00

(52) U.S. Cl. PLT/365

(57) ABSTRACT

A new Anthurium plant particularly distinguished by red, durable spathes that slowly turn brown with age, dark-green, compact, durable leaves, medium-long erect penduncle, more inflorescences than leaves, rich shoot formation, flowers early and freely throughout the year, a compact and full plant habit with a plant height around 30 cm, is disclosed.

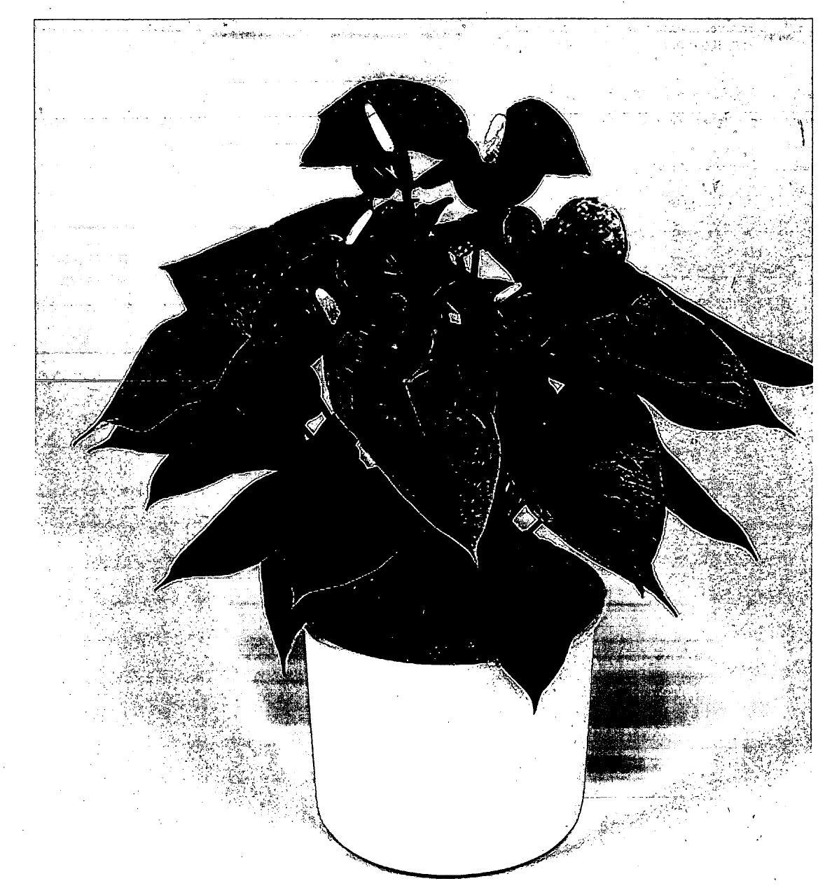

### GENUS AND SPECIES

Anthurium andreanum L.

### VARIETY DENOMINATION

‘Anthepedi’

### BACKGROUND OF THE NEW PLANT

The present invention comprises a new and distinct cultivar of Anthurium, botanically known as Anthurium andreanum L., and hereinafter referred to by the cultivar name ‘Anthepedi’. The new cultivar originated from a hybridization made in 1997 in Bleiswijk, The Netherlands. The female parent was the proprietary pink-color Anthurium pot plant ‘95-634-01’ (unpatented), while the male parent was an unidentified Anthurium plant (unpatented).

The new cultivar was selected in 1999 from the results of the hybridization and has been asexually reproduced repeatedly by tissue culture in Bleiswijk, The Netherlands over a six-year period. The present invention has been found to retain its distinctive characteristics through successive asexual propagations.

Plant Breeder's Rights for this cultivar were applied with the European Union on Mar. 11, 2004 and in Japan on Apr. 30, 2004.

### SUMMARY OF THE INVENTION

The following are the most outstanding and distinguishing characteristics of this new cultivar when grown under normal horticultural practices in Bleiswijk, The Netherlands.
1) Red, durable flowers;
2) Dark-green leaves that are compact and durable;
3) Medium long erect penducle which holds the flowers just above the foliage;
4) Excellent leaf to flower ratio;
5) Rich shoot formation;
6) Compact and full plant habit;
7) Flowers early and rich and is freely flowering throughout the year; and
8) Is a midi-type potplant.

### DESCRIPTION OF PHOTOGRAPH

This new Anthurium plant is illustrated by the accompanying photograph which shows the overall plant habit including blooms, buds and foliage of the plant; the colors shown are as true as can be reasonably obtained by conventional photographic procedures. The photograph is of a 56-week old plant grown in a greenhouse in Bleiswijk, The Netherlands.

### DESCRIPTION OF THE NEW CULTIVAR

The following detailed description sets forth the distinctive characteristics of ‘Anthepedi’. The data which define these characteristics were collected from asexual reproductions carried out in Bleiswijk, The Netherlands. The plant history was taken on 56-week old plants which were planted from tissue culture in 14-cm pots and grown in a glass greenhouse between 19° C. and 24° C. Observations were made in May of 2006. Color readings were taken under 5000 lux natural light in the greenhouse. Color references are primarily to the R.H.S Colour Chart of The Royal Horticultural Society of London (R.H.S) (2001).

### DETAILED BOTANICAL DESCRIPTION

Classification:
   Family.—Aracea.
   Botanical.—Anthurium andreanum L.
   Common.—Anthurium.
Parentage:
   Female parent.—‘95-634-01’, a proprietary pink-flowered Anthurium plant (unpatented).
   Male parent.—An unidentified Anthurium plant (unpatented).
Propagation:
   Propagation.—Tissue culture.
   Root description.—Fleshy-pink to light-brown roots with smaller hairy lateral roots; root-tips are yellow.
   Time to produce a finished flowering plant.—55-60 weeks for a 14 cm pot.
Plant:
   Growth habit.—Herbaceous perennial.
   Height (measured from soil, including inflorescence).—30 cm to 35 cm.
   Width (measured from leaf tips).—30 cm to 35 cm.
Leaves:
   Immature leaves.—Length: 4.0 cm Width: 2.5 cm Color (upper surface): RHS 146A (green) Texture: Very shiny.
   Mature leaves.—Length (fully expanded): 17 cm to 20 cm Width: 11 cm to 13 cm Shape: Elliptical-cordate, weakly cupped Apex: Acuminate Base: Cordate Leaf blade angle with the petiole: Between 110 degrees and 150 degrees Color: Upper surface: RHS 139A (green) Lower surface: RHS 137B (light-green) Texture: Shiny, leathery and thick Venation: The mid-vein and primary veins (the veins which radiate out from junction of petiole and leaf) protrude at the underside of the leaf blade. Venation color: Upper surface: RHS 137C (green) Lower surface: RHS 146D.
   Lobes.—Arrangement: Leaf blade has two small lobes extending past the petiole Length of lobes of mature leaf blades: 17 cm Width of lobes of mature leaf blades: 10 cm Distance for petiole/leaf junction to highest point on lobes of mature leaf blades: 3.5 cm to 4.5 cm.
   Petiole.—Cross-section: Round Diameter: 0.2 cm to 0.3 cm Color: Mature leaf: RHS 137B Immature leaf: RHS 164A Cataphyl color surrounding the petiole: RHS 144B (light-green) with a bit of RHS 179A (reddish) at the tip.
Inflorescence:
   Arrangement.—Single.
   Flowering habit (length of flowering season).—Continuous.
   Number of inflorescences per plant.—4 to 10.
   Fragrance.—Absent.
Spathe:
   Buds.—The spathe is tightly rolled around the spadix and extrudes from the peduncle sheath. After the spathe is fully opened the peduncle elongates some extra centimeters.
   Arrangement.—Spathe angle with the peduncle is between 80 degrees and 100 degrees; the spathe stands on a slightly curved wiry peduncle about 5 cm to 10 cm above the foliage.
   Shape.—Ovate, fully-opened is cupped.
   Apex.—Mucronate tip.
   Base.—Cordate.
   Size.—Height: 35 cm plant Length: 7 cm to 9 cm Width: 9 cm to 10 cm.
   Color.—Just fully open: Upper surface: RHS 53B (red) Lower surface: RHS 53C (red) 7 to 8 weeks after opening: RHS 185A (brownish-red) 14 to 16 weeks after opening: complete flower dies Edge of lobes of spathe (time of year can determine whether the edges of lobes will be green or not): Upper surface: RHS 144A (light-green) Lower surface: RHS 146D (green).
Peduncle:
   Shape.—Erect.
   Cross-section.—Round.
   Length.—20 cm to 25 cm.
   Diameter.—0.3 cm to 0.4 cm.
Flowering time: One small untreated tissue culture plant of 2 cm tall will flower, depending on season. After 15 to 16 months, 2 to 3 blossoms appear. More blossoms appear after some additional weeks so that a full flowering and salable plant will have 4 to 10 red flowers. Smaller blossoms may occur on less mature growth.
Spadix:
   Size.—Height: 2.5 cm to 4 cm Length: 4 cm Width (at apex): 0.5 cm to 0.7 cm Diameter (at base): 0.6 cm to 0.8 cm.
   Shape.—Columnar.
   Color.—Immature: RHS 153D (green-yellow) Mature: RHS 54B (white) Ages to: RHS 141A (green).
Flowers:
   Quantity per spadix.—200 to 300.
   Shape.—Rounded.
   Size.—Length: 0.1 cm to 0.2 cm Diameter (max): 0.2 cm.
   Color.—RHS 156 (white).
Reproductive organs:
   Stamens.—Not visible.
   Pollen.—Sparse.
   Pollen color.—RHS 156 (white).
   Pistil.—Quantity: Sparse Length: Less than 0.01 cm Color: RHS 54B.
   Style.—Not observed.
   Stigma.—Shape: Ovoid Diameter: Less than 0.01 cm Color: RHS 155D (white).
   Ovary.—Rarely visible.
   Ovary color.—RHS 155D (white).
Fruit and seed set: Seed production has not been observed to date.
Disease and insect resistance: No particular resistance or susceptibility different from other Anthurium varieties.

### COMPARISON WITH PARENTAL AND COMMERCIAL CULTIVARS

‘Anthepedi’ differs from the female parent ‘95-634-01’ (unpatented), by having larger, red spathes, while ‘95-634-01’ has smaller, pink spathes. In addition, ‘Anthepedi’ has a less compact plant habit than ‘95-634-01’.

A comparison with the male parent, an unknown Anthurium plant, (unpatented), is not possible because detailed information is unknown.

‘Anthepedi’ is similar to the commercial variety ‘Anthbnena’ (unpatented), however, there are differences as listed in Table 1 below:

TABLE 1
Comparison of characteristics between ‘Anthepedi’ and ‘Anthbnena’

| Characteristic | ‘Anthepedi’ | ‘Anthbnena’ |
|---|---|---|
| Leaf length | 17.0 cm to 20.0 cm | 16.0 cm |
| Leaf width | 11.0 cm to 13.0 cm | 10.0 cm |
| Spathe length | 7.0 cm to 9.0 cm | 7.0 cm |
| Spathe width | 9.0 cm to 10.0 cm | 8.0 cm |
| Spathe durability | Flowers remain on plant for | Flowers remain on plant |
| | over a year | for up to 8 months |

I claim:

1. A new and distinct cultivar of anthurium plant as shown and described herein.
